(19) United States
(12) Patent Application Publication
(10) Pub. No.: US 2015/0086634 A1
(43) Pub. Date: Mar. 26, 2015

(54) COSMETIC USES OF MOLDED PLACENTAL COMPOSITIONS

(71) Applicant: MiMedx Group, Inc., Marietta, GA (US)

(72) Inventors: Thomas J. Koob, Marietta, GA (US); Rebeccah J.C. Brown, Marietta, GA (US)

(21) Appl. No.: 14/480,274

(22) Filed: Sep. 8, 2014

Related U.S. Application Data
(60) Provisional application No. 61/876,191, filed on Sep. 10, 2013.

(51) Int. Cl. A61K 8/98; A61Q 90/00; A61Q 19/08

(52) U.S. Cl. CPC A61K 8/982; A61Q 19/08; A61Q 90/00; A61K 2800/10
USPC 424/489; 424/583

(57) ABSTRACT

Described herein are methods of using molded dehydrated placental compositions, and cosmetic compositions thereof. The compositions have numerous cosmetic applications.

### CROSS-REFERENCE TO RELATED APPLICATION

This application claims the benefit under 35 U.S.C. §119(e) of U.S. Provisional Application No. 61/876,191, filed on Sep. 10, 2013, which is incorporated herein by reference in its entirety.

### BACKGROUND OF THE INVENTION

1. Field of the Invention

This invention is directed to methods for using molded placental compositions and, in particular, methods for using molded dehydrated placental compositions to treat conditions in a patient including cosmetic conditions. Exemplary cosmetic conditions include wrinkle treatments, tissue augmentation, lip and eye augmentation, hair growth, scar inhibition and/or reduction and the like.

2. State of the Art

Placental tissue components such as isolated amnion and chorion, as well as laminates thereof, are known in the art as a basis for wound coverings and wound healing. Typically, the placental tissue is harvested after an elective Cesarean surgery. The placenta is composed of an amniotic membrane which has two primary layers of tissue, amnion and chorion. Amnion tissue is the innermost layer of the amniotic sac and in direct contact with the amniotic fluid. The amniotic sac contains the amniotic fluid and protects the fetal environment. Histological evaluation indicates that the membrane layers of the amnion consist of a single layer of epithelium cells, thin reticular fibers (basement membrane), a thick compact intermediate layer, and a fibroblast cellular layer. The fibrous layer of amnion (i.e., the basement membrane) contains collagen types IV, V, and VII, and cell-adhesion bio-active factors including fibronectin and laminins. Heretofore, wound covering or wound healing compositions comprising placental tissue components typically were in the form of grafts wherein individual layers such as the amnion and/or chorion layer formed one or discreet components of the graft.

While such tissue grafts provide significant medical benefits, these grafts are very thin and lack sufficient structural competency to accord for minimally-invasive means for insertion into an internal organ or body part of a patient.

In addition to their beneficial uses as a tissue graft, placental tissue components, in particular amnion, have been found to act as a stem cell recruiter as provided in U.S. Patent Application Publication No. 2014/0106447, which is incorporated herein by reference in its entirety.

### SUMMARY OF THE INVENTION

Described herein is a unique approach wherein dehydrated micronized placental compositions are molded into shapes and sizes which can be non-invasively introduced into or onto a patient for a variety of cosmetic applications to treat a range of conditions.

This invention is directed, in part, to the discovery that compositions composed of molded, dehydrated micronized placental tissues, such as amnion, chorion, intermediate tissue layer, Wharton's jelly and any combination thereof or laminates thereof, and cosmetic compositions thereof impart significant benefits when used alone or as part of a cosmetic implant. Accordingly, these molded dehydrated micronized placental compositions have numerous cosmetic applications.

In one aspect, the invention relates to novel methods for using molded, dehydrated, placental compositions, such as amnion, chorion, intermediate tissue layer, Wharton's jelly and any combination thereof, or laminates thereof having a defined size and shape. In some embodiments, these compositions comprise amnion and optionally one or more other placental tissue or umbilical cord components such as chorion, intermediate tissue layer, Wharton's jelly and any combination thereof. The placental compositions used in the methods of this invention have a sufficient density and cohesiveness to maintain their size and shape at least until the molded, dehydrated placental compositions are introduced into a subject.

In one embodiment, the density of the molded placental composition, such as an amnion composition or chorion composition, is sufficient such that the placental composition can be formed into any desired shape and the molded placental composition will maintain its shape during introduction into a subject. For example, the density of the placental composition is in a range from at least about 1.2 g/cm³ to about 10 g/cm³ and preferably 2 g/cm³ to about 8 g/cm³. Such molded placental compositions can be introduced using minimally-invasively means into the patient as described herein below or can be introduced by conventional invasive means. This permits the attending clinician significant flexibility in treating the patient taking into account the purpose of the treatment, the age and condition of the patient, and other factors well within the skill of the art.

In another embodiment, the molded, dehydrated placental composition is sufficiently cohesive such that the placental composition does not break, splinter, disintegrate or fragment during introduction into a subject. The suitable density and cohesiveness of the molded composition can be determined by one skilled in the art based on the purpose for introduction, the amount of placental composition to be used, the manner of administration/introduction, and the specific body part for administration. The desired shape of the molded, dehydrated placental composition is maintained at least until introduction into a subject.

The molded, dehydrated placental composition is designed to erode after introduction wherein the period for erosion is defined by the density and degree of cohesiveness of the molded, dehydrated amnion as well as its size. During erosion, growth factors and other biological factors are released over time from the placental composition, thereby providing for sustained release of such factors at the location of administration/introduction.

In a related aspect, the molded, dehydrated placental composition with a defined size and shape is obtained by compressing one or more dehydrated micronized placental tissue component(s), and optionally a filler, or any combination thereof, under a suitable compressive pressure, e.g., a pressure ranging from about 1 to about 5000 MPa, for a defined period of time, e.g., from about 10 seconds to about 10 minutes, or longer, such that the obtained molded dehydrated micronized composition will possess sufficient density and cohesive mass to maintain its size and shape at least until administered or implemented. Preferred pressures include from about 10 to 1000 MPa or from about 20 to about 500 MPa. Preferred times include from about 0.5 to 9 minutes or from 1 to 5 minutes.

Prior to compression, the micronized placental composition is dehydrated to the extent that compression can be performed in a non-porous mold. In other words, the water content in the dehydrated micronized placental composition is sufficiently low that compression to form the compositions of this invention can be conducted in a non-porous mold. For instance, the water content of the dehydrated micronized placental composition is preferably less than about 20%, less than about 15%, less than about 10%, or less than about 5%.

It is within the purview of one of ordinary skill in the art to use a suitable means to compress the micronized placental composition to achieve the desired density and cohesiveness. For instance, the micronized placental composition may be compressed through use of a pressure mold of any desired size and shape to obtain the desired size and shape.

It is within the purview of one skilled in the art to choose any molding technique where the dehydrated micronized placental compositions are loaded into a shaped mold and subjected to a pressure such that the placental composition takes the shape of the mold following compression. The molded placental composition can take any desired shape, such as the shapes of pins, nails, barrels, rivets, darts, and membranes. In certain embodiments, the resulting molded, dehydrated placental composition can have a hollow center or voids in the graft. Voids in the molded composition can be achieved by use of a laser to induce channels into or through the composition. One skilled in the art would understand that once the molded placental composition is formed, it can be subjected to post-formation treatment, such as cavitation by laser drilling, to increase the surface area of the molded placental composition.

In one embodiment, the compression of dehydrated micronized placental composition is carried out under controlled temperature to minimize denaturation of proteins, growth factors and other biological factors contained in the composition. As molding often raises the temperature within the mold, a heat sink can be employed in conjunction with the molding process to control the temperature increase.

This invention is specifically directed to methods for using molded placental compositions. In one embodiment, the molded placental compositions are in the form of fine needles which can be inserted directly into a body part or under the skin. For example, direct insertion of the molded placental composition into an area adjacent a torn ligament or tendon will provide significant quantities of growth factors to facilitate healing. The torn ligament or tendon can be located in the ankle, knee, elbow, wrist, shoulder and the like. In another embodiment, such fine needles can be inserted under the skin such as under a wrinkle to provide both mass to limit or eliminate the wrinkle while providing growth factors to promote growth of tissue, collagen, etc. such that as the placental mass dissipates, it is replaced at least in part by in situ growth of tissue which provides for long term wrinkle removal. Still further, the molded placental compositions can be used to reduce or inhibit scarring, provide for lip augmentation, as well as during plastic surgery to promote healing and reduce scarring. Still further, the molded placental compositions can be used to augment tissue such as in the cheeks so as to provide a fuller facial appearance.

The present invention provides a cosmetic method for altering the appearance of a patient which method comprises identifying a perceived defect in the patient and altering said defect by placing a placental composition in or adjacent said defect in a manner to alter the appearance of the defect, wherein the placental composition comprises molded, micronized amnion, and/or molded, micronized chorion. Also provided is the above method, wherein said cosmetic method is selected from the group consisting of a wrinkle reduction, lip augmentation, collagen replacement treatment, and facial augmentation. In another aspect, what is provided is the above method, wherein said placental composition comprises molded micronized amnion and optionally micronized biocompatible polymer. Also contemplated is the above method, wherein said micronized biocompatible polymer is a plasticizing polymer.

In a composition embodiment, what is provided is a molded placental composition comprising micronized placental tissue which further comprises a biocompatible plasticizing polymer. In another aspect, what is provided is the above molded placental composition, wherein the micronized placental tissue is cross-linked with a biocompatible cross-linking agent. Also provided is the above molded placental composition, wherein the plasticizing polymer is cross-linked with a biocompatible cross-linking agent.

In a cosmetic method embodiment, the present invention provides a cosmetic method for altering the appearance of a patient which method comprises administering a molded placental composition to a subject, wherein said molded placental composition has a defined size and shape comprising micronized placental tissue, and wherein said composition has a sufficient density and cohesiveness to maintain its size and shape for a defined period of time ex vivo and in vivo.

In another methods embodiment, what is provided is a method for producing a molded placental composition having a preselected disintegration rate and/or strength, said method comprising adjusting the compression force, compression rate, density and/or particle size such that the resulting molded placental composition has the preselected disintegration rate and/or strength. Moreover, the invention provides the above method, wherein the particle size is greater than about 250 μm. Furthermore, what is provided is the above method, wherein the particle size is between about 75 μm and about 150 μm, and the above method, wherein the particle size is less than about 75 μm.

In yet another methods embodiment, the present invention encompasses a method for producing a molded placental composition having a preselected strength and/or stiffness, said method comprising adjusting the compression force, compression rate, density and/or particle size such that the resulting molded placental composition has the preselected strength and/or stiffness.

Several of the advantages of this invention are set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawing(s), which are incorporated in and constitute a part of this specification, illustrate several aspects described below.

FIG. 1 is an overview flow chart of the process for making the molded, dehydrated amnion composition described herein.

FIG. 2 represents the Young's Modulus and stress at failure for molded, micronized amnion compositions made with particle sizes of greater than about 250 μm, between about 75 μm and about 150 μm, and less than about 75 μm approximate diameter.

FIG. 3A represents the Young's Modulus and stress at failure for molded, micronized amnion compositions that were formed using the indicated compression forces.

FIG. 3B represents the Young's Modulus and stress at failure for molded, micronized amnion compositions that were formed using the indicated compression rates.

FIG. 3C represents the Young's Modulus and stress at failure for molded, micronized amnion compositions that were formed using the indicated number of compression cycles.

### DETAILED DESCRIPTION

Before this invention is disclosed and described, it is to be understood that the aspects described below are not limited to specific compositions, methods or preparing such compositions, or uses thereof as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a bioactive agent” includes mixtures of two or more such agents, and the like.

“Optional” or “optionally” means that the subsequently described event or circumstance can or cannot occur, and that the description includes instances where the event or circumstance occurs and instances where it does not. For example, the phrase “optionally cleaning step” means that the cleaning step may or may not be performed.

The term “subject” as used herein is any vertebrate organism including but not limited to mammalian subjects such as humans, farm animals, domesticated pets and the like.

The term “amnion” as used herein includes amniotic membrane where the intermediate tissue layer is intact or has been substantially removed.

The term “amnion composition” as used herein refers to a dehydrated, micronized composition comprising amnion. The composition may optionally comprise one or more other placental tissue components, and/or other factors (e.g., plasticizer, bioactive agents, fillers, etc.) as described herein or as would be apparent to one of skill in the art.

The term “filler” refers to any component of the composition other than amnion. Filler includes other placental tissue components as well as polymers, including collagen, hyaluronic acid, biocompatible plasticizers and the like. In one embodiment, the collagen includes human collagen, a collagen derivative, a human collagen derivative, and collagen prepared from placental tissue, such as collagen materials substantially free of non-human antigens. In some embodiments, the collagen is prepared form the fibrous layer of amnion (i.e., the basement membrane) which contains collagen types IV, V, and VII. The collagen filler described herein is separate and apart from the collagen, if any, that exists in the placental component, such as in the amnion or chorion, of the composition. In some aspects, the collagen is free or substantially free of other components, including elastin, fibronectin, and/or laminin.

The term “non-human antigen” refers to any agent (e.g., protein, peptide, polysaccharide, glycoprotein, glycolipid, nucleic acid, or combination thereof) of non-human origin that, when introduced into a human, is immunogenic, eliciting an unwanted immune response that requires medical treatment for the manifestations (e.g., inflammation, etc.) of the immune response. As defined herein, the non-human antigen-induced immune response can be humoral or cell-mediated, or both.

The term “placental tissue” or “placental tissue components” refers to any and all of the well-known components of the placenta and optionally the umbilical cord including but not limited to amnion, chorion, intermediate tissue layer, an umbilical cord component such as Wharton's Jelly, and the like. In one preferred embodiment, the placental tissue includes umbilical cord components and, in another embodiment, such umbilical cord components (e.g., Wharton's jelly, umbilical cord vein and artery, and surrounding membrane) are not included.

The term “mold,” “molded,” or “molding” includes any form of pressure molding such as the use of actual molds, extrusion under pressure, stamping or any pressurized method, and the like such that micronized placental tissue, and optionally filler, are compressed under pressure to produce a molded placental composition that has a defined size, shape, density and cohesiveness for a defined period of time for use either ex vivo or in vivo.

The term “comprising” means any recited elements are necessarily included and other elements may optionally be included. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

The term “subject” or “patient” as used herein refers to any vertebrate organism including, but not limited to, mammalian subjects such as humans, farm animals, domesticated pets and the like.

The term “biocompatible” as used herein refers to a material that is suitable for implantation or injection into a subject. In various aspects, a biocompatible material does not cause toxic or injurious effects once implanted in the subject.

The term “modified placental tissue” refers to any and all components of placental tissue including whole placental tissue that has been modified by cleaning, disinfecting, and/or segmenting the tissue as well as to separated components of placental tissue such as amnion, chorion, the umbilical cord, and the like. Modified tissue may maintain cellular layers, such as the epithelial layer and/or the fibroblast layer. Modified placental tissue may include further modification, such as lamination of one or more layers of placental tissue, micronization of placental tissue, chemisorption or physisorption of small molecules, proteins (e.g. growth factors, antibodies), nucleic acids (e.g. aptamers), polymers, or other substances.

The term “sufficient amount of” refers to an amount of a composition that is sufficient to have the desired effect, e.g. provoke stem cell recruitment proximate to or on the composition over time, either in vivo or in vitro. The “sufficient amount” of an placental composition, such as an amnion or chorion composition will vary depending on a variety of factors, such as but not limited to, the type and/or amount of placental composition used, the type and/or amount of filler used, the type and/or size of the intended organ and/or body part to be treated, the severity of the disease or injury to the organ and/or body part to be treated and the administration route. The determination of a “sufficient amount” can be made by one of ordinary skill in the art based on the disclosure provided herein.

The term “stem cell recruiting factors” refers to any and all factors that are capable of recruiting stem cells and causing them to migrate towards a source of such factors. Non-limiting examples of stem cell recruiting factors may be one or more CC chemokines, CXC chemokines, C chemokines, or CX3C chemokines.

The term “stem cell recruitment” refers to direct or indirect chemotaxis of stem cells to a placental composition, such as an amnion or chorion composition. The recruitment may be direct, wherein stem cell recruiting factors (e.g. chemokines, which induce cell chemotaxis) in the composition are released from the composition and induce stem cells to migrate towards the amnion composition. In one aspect, the recruitment may be indirect, wherein stem cell recruiting factors in the composition are released from the composition which induce nearby cells to release factors (e.g. chemokines), that in turn induce stem cells to migrate towards the composition. Still further, stem cell recruitment may embody both direct and indirect factors.

The term “diseased” as used herein refers to an organ and/or body part that is characterized as being in a disease state, or susceptible to being in a disease state, wherein the disease is amenable to treatment with stem cells.

The term “injured” as used herein is used to have an ordinary meaning in the art, and includes any and all types of damage to an organ and/or body part, wherein the injury is amenable to treatment with stem cells.

The term “implantable” and derivatives thereof means the device can be inserted, embedded, grafted or otherwise acutely or chronically attached or placed in or on a subject.

Titles or subtitles may be used in the specification for the convenience of a reader, which are not intended to influence the scope of the present invention. Additionally, some terms used in this specification are more specifically defined below.

The term “treatment” or “treating”, to the extent it relates to a disease or condition, includes preventing the disease or condition from occurring or reoccurring, inhibiting development of the disease or condition, reducing or eliminating the disease or condition, and/or relieving one or more symptoms of the disease or condition.

Titles or subtitles may be used in the specification for the convenience of a reader, which are not intended to influence the scope of the present invention. Additionally, some terms used in this specification are more specifically defined below.

### I. Compositions Comprising Molded Dehydrated Micronized Placental Compositions

Described herein are molded dehydrated placental compositions and cosmetic compositions thereof. Such compositions are prepared from micronized amnion, described in International Patent Application WO2012/112410, as well as in U.S. provisional application Ser. Nos. 61/442,346, 61/543,995, and US2014/0050788. The contents of these applications are specifically incorporated by reference in their entireties. It is understood that the term “micronized” is meant to include micron and sub-micron sized particles.

In one aspect, the invention is directed to a composition that includes (a) a molded dehydrated micronized placental composition, and optionally (b) a biologically acceptable excipient including biologically acceptable fillers, cosmetically acceptable components and the like. Such fillers and/or components are employed to enhance one or more properties of the molded compositions such as color, lubricity, cohesiveness, stability, anti-oxidant properties, flexibility, and degradation rate.

For example, the invention includes a molded, dehydrated placental composition which can be used without the addition of any fillers, stabilizers, buffers, or cosmetic components. Alternatively, the invention includes molded, dehydrated placental composition and at least one excipient such as a filler, a stabilizer, a buffer, a coloring agent, a disintegrating agent and the like and optionally one or more cosmetic components. Preferably, the coloring agent facilitates locating and properly placing the placental composition, which is otherwise almost transparent, to the intended treatment site. The disintegrating agent modifies the rate that the molded dehydrated placental composition erodes or disintegrates in vivo after being introduced to a subject.

The compositions preferably comprise micronized placental composition, for example, micronized amnion and/or micronized chorion. In yet another aspect, the composition includes molded, dehydrated placental comprising micronized placenta. In yet another aspect, the composition includes molded, dehydrated placental comprising micronized placenta, which epithelium layer is substantially removed from the placenta. Moreover, the composition includes molded, dehydrated amnion comprising micronized amnion. In yet another aspect, the composition includes molded, dehydrated amnion comprising micronized amnion, which epithelium layer is substantially removed from the amnion.

As one of ordinary skill in the art would understand, the molded, dehydrated placental composition of the invention is compressed under pressure into any shape or size, as long as the molded placental composition has a cohesive mass having a defined density.

Alternatively, the dehydrated micronized placental composition is compressed into any mold having a desired shape or size such that the molded dehydrated placental composition takes the shape and size of the mold and exhibiting the desired cohesiveness and density. It is within the purview of one of ordinary skill in the art to select suitable molding material, such as silicone, resin, Teflon®, or stainless steel, to form a mold of desired shape and size.

Advantageously, the molded, dehydrated placental composition described herein has low water resorbability such that the shape of the placental composition is maintained at least through the period of introduction into the patient and preferably for a defined period of time in vivo following introduction to a subject. It is understood that the placental composition erodes in vivo and releases the growth factors and other biological factors contained in the placental composition. Thus, the retention of the shape of the molded, dehydrated placental composition means that the overall shape is retained even if that shape is subject to or initiates erosion.

### II. Methods of Making Compositions Comprising Molded Dehydrated Placental Components

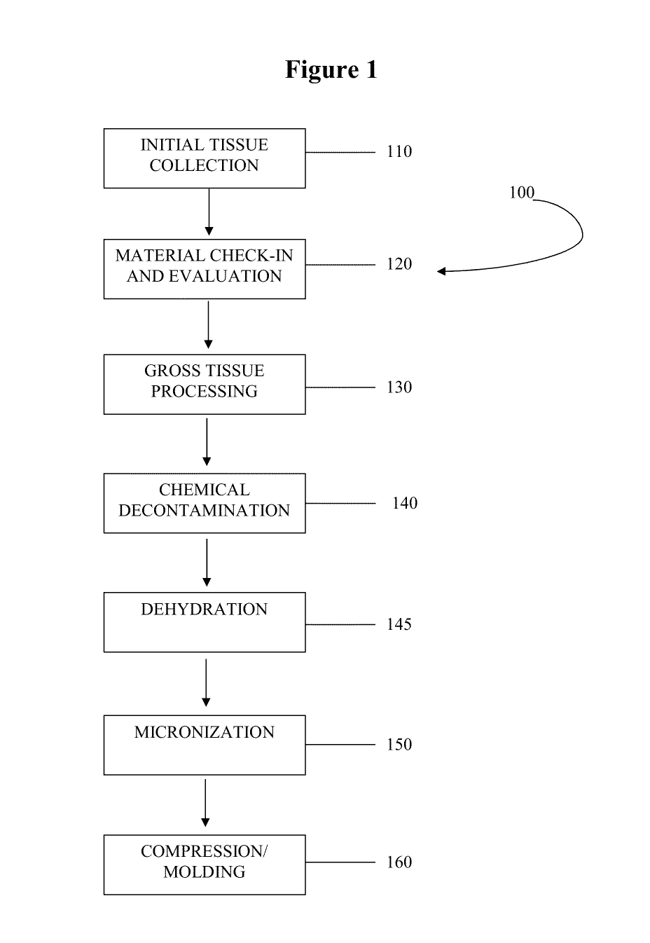

FIG. 1 depicts an overview (100) and certain aspects of the steps to harvest, process, and prepare molded dehydrated micronized placental material, e.g. amnion. More detailed descriptions and discussion regarding each individual step will follow. Initially, the placental tissue is collected from a consenting patient following an elective Cesarean surgery (step 110). The material is preserved and transported in conventional tissue preservation manner to a suitable processing location or facility for check-in and evaluation (step 120). Gross processing, handling, and separation of the tissue layers then takes place (step 130). Acceptable tissue is then decontaminated (step 140) and dehydrated (step 145). After decontamination and dehydration, the amnion composition components (e.g., amnion, intermediate tissue layer and/or chorion individually or as grafts) are then micronized (step 150). The micronized components are compressed/molded under pressure into a desired shape or size (step 160). Each step is described in detail below.

### Initial Tissue Collection (Step 110)

The components used to produce the molded, dehydrated amnion composition described herein are derived from the placenta. The source of the placenta can vary. In one aspect, the placenta is derived from a mammal such as human and other animals including, but not limited to, cows, pigs, and the like can be used herein. In the case of humans, the recovery of the placenta originates in a hospital, where it is preferably collected during a Cesarean section birth. The donor, referring to the mother who is about to give birth, voluntarily submits to a comprehensive screening process designed to provide the safest tissue possible for transplantation. The screening process preferably tests for antibodies to human immunodeficiency virus type 1 and type 2 (anti-HIV-1 and anti-HIV-2), antibodies to the hepatitis B virus (anti-HBV), hepatitis B surface antigens (HBsAg), antibodies to the hepatitis C virus (anti-HCV), antibodies to the human T-lymphotropic virus type I and type II (anti-HTLV-I, anti-HTLV-II), CMV, and syphilis, and nucleic acid testing for human immune-deficiency virus type 1 (HIV-1) and for the hepatitis C virus (HCV), using conventional serological tests. The above list of tests is exemplary only, as more, fewer, or different tests may be desired or necessary over time or based upon the intended use of the placental compositions, as will be appreciated by those skilled in the art.

Based upon a review of the donor's information and screening test results, the donor will either be deemed acceptable or not. In addition, at the time of delivery, cultures are taken to determine the presence of bacteria, for example, Clostridium or Streptococcus. If the donor's information, screening tests, and the delivery cultures are all satisfactory (i.e., do not indicate any risks or indicate acceptable level of risk), the donor is approved by a medical director and the tissue specimen is designated as initially eligible for further processing and evaluation.

Human placentas that meet the above selection criteria are preferably bagged in a saline solution in a sterile shipment bag and stored in a container of wet ice for shipment to a processing location or laboratory for further processing.

If the placenta is collected prior to the completion of obtaining the results from the screening tests and delivery cultures, such tissue is labeled and kept in quarantine. The placenta is approved for further processing only after the required screening assessments and delivery cultures, which declare the tissue safe for handling and use, are satisfied and final approval is obtained from a medical director.

### Material Check-In and Evaluation (Step 120)

Upon arrival at the processing center or laboratory, the shipment is opened and verified that the sterile shipment bag/container is still sealed and in the coolant, that the appropriate donor paperwork is present, and that the donor number on the paperwork matches the number on the sterile shipment bag containing the tissue. The sterile shipment bag containing the tissue is then stored in a refrigerator until ready for further processing.

Gross Tissue Processing (Step 130)

When the tissue is ready to be processed further, the sterile supplies necessary for processing the placental tissue further are assembled in a staging area in a controlled environment and are prepared for introduction into a controlled environment. In one aspect, the placenta is processed at room temperature. If the controlled environment is a manufacturing hood, the sterile supplies are opened and placed into the hood using conventional sterilization techniques. If the controlled environment is a clean room, the sterile supplies are opened and placed on a cart covered by a sterile drape. All the work surfaces are covered by a piece of sterile drape using conventional sterilization techniques, and the sterile supplies and the processing equipment are placed onto the sterile drape, again using conventional sterilization techniques.

Processing equipment is decontaminated according to conventional and industry-approved decontamination procedures and then introduced into the controlled environment. The equipment is strategically placed within the controlled environment to minimize the chance for the equipment to come in proximity to or be inadvertently contaminated by the tissue specimen.

Next, the placenta is removed from the sterile shipment bag and transferred aseptically to a sterile processing basin within the controlled environment. The sterile basin contains hypertonic saline solution (e.g., 18% NaCl) that is at or near room temperature. The placenta is gently massaged to help separate blood clots and to allow the placental tissue to reach room temperature, which facilitates the separation of the placental components from each other (e.g., amnion membrane and chorion). After having warmed up to ambient temperature (e.g., after about 10-30 minutes), the placenta is then removed from the sterile processing basin and laid flat on a processing tray with the amnion membrane layer facing down for inspection.

The placenta is examined for discoloration, debris or other contamination, odor, and signs of damage. The size of the tissue is also noted. A determination is made, at this point, as to whether the tissue is acceptable for further processing.

The amnion and chorion are next carefully separated. In one aspect, the materials and equipment used in this procedure include a processing tray, 18% saline solution, sterile 4×4 sponges, and two sterile Nalgene jars. The placenta tissue is then closely examined to find an area (typically a corner) in which the amnion can be separated from the chorion. The amnion appears as a thin, opaque layer on the chorion.

The fibroblast layer is identified by gently contacting each side of the amnion with a piece of sterile gauze or a cotton tipped applicator. The fibroblast layer will stick to the test material. The amnion is placed into processing tray basement membrane layer down. Using a blunt instrument, a cell scraper, or sterile gauze, any residual blood is also removed. This step must be done with adequate care, again, so as not to tear the amnion. The cleaning of the amnion is complete once the amnion is smooth and opaque-white in appearance.

In certain aspects, the intermediate tissue layer, also referred to as the spongy layer, is substantially removed from the amnion in order to expose the fibroblast layer. The term “substantially removed” with respect to the amount of intermediate tissue layer removed is defined herein as removing greater than about 90%, greater than about 95%, or greater than about 99% of the intermediate tissue layer from the amnion. This can be performed by peeling the intermediate tissue layer from the amnion. Alternatively, the intermediate tissue layer can be removed from the amnion by wiping the intermediate tissue layer with gauze or other suitable wipe. The resulting amnion can be subsequently decontaminated using the process described below.

In certain aspects, the epithelium layer present on the amnion is substantially removed in order to expose the basement layer of the amnion. The term “substantially removed” with respect to the amount of epithelium removed is defined herein as removing greater than about 90%, greater than about 95%, or greater than about 99% of the epithelial cells from the amnion. The presence or absence of epithelial cells remaining on the amnion layer can be evaluated using techniques known in the art. For example, after removal of the epithelial cell layer, a representative tissue sample from the processing lot is placed onto a standard microscope examination slide. The tissue sample is then stained using Eosin Y Stain and evaluated as described below. The sample is then covered and allowed to stand. Once an adequate amount of time has passed to allow for staining, visual observation is done under magnification.

The epithelium layer can be removed by techniques known in the art. For example, the epithelium layer can be scraped off of the amnion using a cell scraper. Other techniques include, but are not limited to, freezing the membrane, physical removal using a cell scraper, or exposing the epithelial cells to nonionic detergents, anionic detergents, and nucleases. The de-epithelialized tissue is then evaluated to determine that the basement membrane has not been compromised and remains intact. This step is performed after completion of the processing step and before the tissue has been dehydrated, as described in the next section. For example, a representative sample graft is removed for microscopic analysis. The tissue sample is place onto a standard slide, stained with Eosin Y and viewed under the microscope. If epithelium is present, it will appear as cobblestone-shaped cells.

The methods described herein do not remove all cellular components in the amnion. This technique is referred to in the art as “decellularization.” Decellularization generally involves the physical and/or chemical removal of all cells present in the amnion, which includes epithelial cells and fibroblast cells. For example, although the removal of epithelial cells is optional, the fibroblast layer present in the amnion stromal layer is intact, even if the intermediate tissue layer is removed. Here, fibroblast cells are present in the fibroblast layer.

When the placental tissue is Wharton's jelly, the following exemplary procedure can be used. Using a scalpel or scissors, the umbilical cord is dissected away from the chorionic disk. Once the veins and the artery have been identified, the cord is dissected lengthwise down one of the veins or the artery. Once the umbilical cord has been dissected, surgical scissors and forceps can be used to dissect the vein and artery walls from the Wharton's jelly. Next, the outer layer of amnion is removed from the Wharton's jelly by cutting the amnion. Here, the outer membrane of the umbilical cord is removed such that Wharton's jelly is the only remaining component. Thus, the Wharton's jelly as used herein does not include the outer umbilical cord membrane and umbilical cord vessels. The Wharton's jelly can be cut into strips. In one aspect, the strips are approximately 1-4 cm by 10-30 cm with an approximate thickness of about 1.25 cm; however, other thicknesses are possible depending on the application.

### Chemical Decontamination (Step 140)

The placental tissue can be chemically decontaminated using the techniques described below. In one aspect, the amnion is decontaminated at room temperature. In one aspect, the amnion produced in step 130 (e.g., with or without the intermediate tissue layer) can be placed into a sterile Nalgene® jar for the next step. In one aspect, the following procedure can be used to clean the amnion. A Nalgene jar is aseptically filled with 18% saline hypertonic solution and sealed (or sealed with a top). The jar is then placed on a rocker platform and agitated for between 30 and 90 minutes, which further cleans the amnion of contaminants. If the rocker platform was not in the critical environment (e.g., the manufacturing hood), the Nalgene jar is returned to the controlled/sterile environment and opened. Using sterile forceps or by aseptically decanting the contents, the amnion is gently removed from the Nalgene jar containing the 18% hypertonic saline solution and placed into an empty Nalgene jar. This empty Nalgene jar with the amnion is then aseptically filled with a pre-mixed antibiotic solution. In one aspect, the premixed antibiotic solution is composed of a cocktail of antibiotics, such as Streptomycin Sulfate and Gentamicin Sulfate. Other antibiotics, such as Polymixin B Sulfate and Bacitracin, or similar antibiotics now available or available in the future, are also suitable. Additionally, it is preferred that the antibiotic solution be at room temperature when added so that it does not change the temperature of or otherwise damage the amnion. This jar or container containing the amnion and antibiotics is then sealed or closed and placed on a rocker platform and agitated for, preferably, between 60 and 90 minutes. Such rocking or agitation of the amnion within the antibiotic solution further cleans the tissue of contaminants and bacteria. Optionally, the amnion can be washed with a detergent. In one aspect, the amnion can be washed with 0.1 to 10%, 0.1 to 5%, 0.1 to 1%, or 0.5% Triton-X® wash solution.

If the rocker platform was not in the critical environment (e.g., the manufacturing hood), the jar or container containing the amnion and antibiotics is then returned to the critical/sterile environment and opened. Using sterile forceps, the amnion is gently removed from the jar or container and placed in a sterile basin containing sterile water or normal saline (0.9% saline solution). The amnion is allowed to soak in place in the sterile water/normal saline solution for at least 10 to 15 minutes. The amnion may be slightly agitated to facilitate removal of the antibiotic solution and any other contaminants from the tissue. After at least 10 to 15 minutes, the amnion is ready to be dehydrated and processed further.

In the case of chorion, the following exemplary procedure can be used. After separation of the chorion from the amnion and removal of clotted blood from the fibrous layer, the chorion is rinsed in 18% saline solution for 15 minutes to 60 minutes. During the first rinse cycle, 18% saline is heated in a sterile container using a laboratory heating plate such that the solution temperature is approximately 48° C. The solution is decanted, the chorion tissue is placed into the sterile container, and decanted saline solution is poured into the container. The container is sealed and placed on a rocker plate and agitated for 15 minutes to 60 minutes. After 1 hour agitation bath, the chorion tissue is removed and placed into second heated agitation bath for an additional 15 minutes to 60 minutes rinse cycle. Optionally, the chorion tissue can be washed with a detergent (e.g., Triton-X wash solution) as discussed above for the decontamination of amnion. The container is sealed and agitated without heat for 15 minutes to 120 minutes. The chorion tissue is next washed with deionized water (250 ml of DI water×4) with vigorous motion for each rinse. The tissue is removed and placed into a container of 1× phosphate buffered saline (PBS) w/EDTA solution. The container is sealed and agitated for 1 hour at controlled temperature for 8 hours. The chorion tissue is removed and rinsed using sterile water. A visual inspection was performed to remove any remaining discolored fibrous blood material from the chorion tissue. The chorion tissue should have a cream white visual appearance with no evidence of brownish discoloration.

The following exemplary procedure can be used when the placental tissue is Wharton's jelly. The Wharton's jelly is transferred to a sterile Nalgene jar. Next, room temperature 18% hypertonic saline solution is added to rinse the tissue and the jar is sealed. The jar is agitated for 30 to 60 minutes. After incubation, the jar is decontaminated and returned to the sterile field. The tissue is transferred to a clean sterile Nalgene jar and prewarmed (about 48° C.) with 18% NaCl. The container is sealed and placed on rocker plate and agitated for 60 to 90 minutes.

After the rinse, the jar is decontaminated and returned to the sterile field. The tissue is removed and placed into an antibiotic solution. The container is sealed and agitated for 60 to 90 minutes on a rocker platform. Following incubation, the jar may be refrigerated at 1° C. to 10° C. for up to 24 hours.

The Wharton's jelly is next transferred to a sterile basin containing approximately 200 mL of sterile water. The tissue is rinsed for 1-2 minutes and transferred to a sterile Nalgene jar containing approximately 300 ml of sterile water. The jar is sealed and placed on the rocker for 30 to 60 minutes. After incubation, the jar is returned to the sterile field. The Wharton's jelly should have a cream white visual appearance with no evidence of brownish discoloration.

### Dehydration (Step 145)

In one aspect, the placental tissue or components thereof as described above, or any combination thereof, can be processed into tissue grafts (i.e., laminates) that are subsequently micronized. In another aspect, the placental tissue or individual components thereof can be dehydrated independently and subsequently micronized alone or as a mixture of components. In one aspect, the tissue (i.e., individual membrane or graft) is dehydrated by chemical dehydration followed by freeze-drying. In one aspect, the chemical dehydration step is performed by contacting the amnion, chorion, and/or intermediate layer with a polar organic solvent for a sufficient time and amount in order to substantially (i.e., greater than 90%, greater than 95%, or greater than 99%) or completely remove residual water present in the tissue (i.e., dehydrate the tissue). The solvent can be protic or aprotic. Examples of polar organic solvents useful herein include, but are not limited to, alcohols, ketones, ethers, aldehydes, or any combination thereof. Specific, non-limiting examples include dimethylsulfoxide (DMSO), acetone, tetrahydrofuran, ethanol, isopropanol, or any combination thereof. In one aspect, the placental tissue is contacted with a polar organic solvent at room temperature. No additional steps are required, and the tissue can be freeze-dried directly as discussed below.

After chemical dehydration, the tissue is freeze-dried in order to remove any residual water and polar organic solvent. In one aspect, the amnion, chorion, and/or intermediate layer can be laid on a suitable drying fixture prior to freeze-drying. For example, one or more strips of amnion can be laid on a suitable drying fixture. Next, chorion is laid on top of the amnion. In this aspect, an amnion/chorion tissue graft is produced. Alternatively, a strip of amnion can be placed on a first drying fixture, and a strip of chorion can be placed on a second drying fixture. The drying fixture is preferably sized to be large enough to receive the placental tissue, fully, in laid out, flat fashion. In one aspect, the drying fixture is made of Teflon® or of Delrin®, which is the brand name for an acetal resin engineering plastic invented and sold by DuPont and which is also available commercially from Werner Machine, Inc. in Marietta, Ga. Any other suitable material that is heat and cut resistant, capable of being formed into an appropriate shape to receive wet tissue can also be used for the drying fixture.

Once the tissue is placed on the drying fixture, the drying fixture is placed in the freeze-dryer. The use of the freeze-dryer to dehydrate the tissue can be more efficient and thorough compared to other techniques such as thermal dehydration. In general, it is desirable to avoid ice crystal formation in the placental tissue as this may damage the extracellular matrix in the tissue. By chemically dehydrating the placental tissue prior to freeze-drying, this problem can be avoided.

In another aspect, the dehydration step involves applying heat to the tissue. In one aspect, the amnion, chorion, and/or intermediate layer is laid on a suitable drying fixture (either as individual strips or as a laminate discussed above), and the drying fixture is placed in a sterile Tyvex (also called Tyvek®; or similar, breathable, heat-resistant, and sealable material) dehydration bag and sealed. The breathable dehydration bag prevents the tissue from drying too quickly. If multiple drying fixtures are being processed simultaneously, each drying fixture is either placed in its own Tyvex bag or, alternatively, placed into a suitable mounting frame that is designed to hold multiple drying frames thereon and the entire frame is then placed into a larger, single sterile Tyvex dehydration bag and sealed.

The Tyvex dehydration bag containing the one or more drying fixtures is then placed into a non-vacuum oven or incubator that has been preheated to approximately 35 to 50° C. The Tyvex bag remains in the oven for between 30 to 120 minutes. In one aspect, the heating step can be performed for 45 minutes at a temperature of approximately 45° C. to dry the tissue sufficiently but without over-drying or burning the tissue. The specific temperature and time for any specific oven will need to be calibrated and adjusted based on other factors including altitude, size of the oven, accuracy of the oven temperature, material used for the drying fixture, number of drying fixtures being dried simultaneously, whether a single or multiple frames of drying fixtures are dried simultaneously, and the like.

In one aspect, the placental tissue or other components can be dehydrated using a dehydration device which enhances the rate and uniformity of the dehydration process. Representative dehydration devices suitable for drying placental tissue grafts are described in U.S. Patent Application Publication Nos. US2014/0050788, and in US2014/0051059. The contents of these applications are incorporated by reference in their entireties.

### Preparation of Micronized Placental Components (Step 150)

Once the placental tissue or components thereof as described above, and optionally other components of the amnion compositions, have been dehydrated individually or in the form of tissue graft, the dehydrated tissue(s) and/or other component(s) is micronized. The micronized components can be produced using instruments known in the art. For example, the Retsch Oscillating Mill MM400 can be used to produce the micronized compositions described herein. The particle size of the materials in the micronized composition can vary as well depending upon the application of the micronized composition. In one aspect, the micronized composition has particles that are less than about 500 μm, less than about 400 μm, less than about 300 μm, less than about 200 μm, less than about 100 μm, less than about 75 μm, less than about 50 μm, less than about 25 μm, less than about 20 μm, less than about 15 μm, less than about 10 μm, less than about 9 μm, less than about 8 μm, less than about 7 μm, less than about 6 μm, less than about 5 μm, less than about 4 μm, less than about 3 μm, less than about 2 μm, or from about 2 μm to about 400 μm, from about 25 μm to about 300 μm, from about 25 μm to about 200 μm, from about 25 μm to about 150 μm, or from about 75 μm to about 150 μm. In one aspect, the micronized composition has particles that have a diameter less than about 150 μm, less than about 100 μm, less than about 75 μm, or less than about 50 μm. In other aspects, particles having a larger diameter (e.g. greater than about 250 μm or about 150 μm to about 350 μm) are desirable. In all cases, the diameter of the particle is measured along its longest axis. Particle size is defined as the average size of the particles in the composition.

In one embodiment, the size of the particles may be reduced to nano-range. As one skilled in the art would understand, nanoparticles of placental components may be desirable for the increased density and/or increased release rate upon applying to the wound. Preferably, the particle size of the micronized particles is from about 0.05 μm to about 2 μm, from about 0.1 μm to about 1.0 μm, from about 0.2 μm to about 0.8 μm, from about 0.3 μm to about 0.7 μm, or from about 0.4 μm to about 0.6 μm. Alternatively, the particle size of the micronized particles is at least about 0.05 μm, at least about 0.1 μm, at least about 0.2 μm, at least about 0.3 μm, at least about 0.4 μm, at least about 0.5 μm, at least about 0.6 μm, at least about 0.7 μm, at least about 0.8 μm, at least about 0.9 μm, or at least about 1 μm. Alternatively, the particle size of the micronized particles is less than about 1 μm, less than about 0.9 μm, less than about 0.8 μm, less than about 0.7 μm, less than about 0.6 μm, less than about 0.5 μm, less than about 0.4 μm, less than about 0.3 μm, less than about 0.2 μm, less than about 0.1 μm, or less than about 0.05 μm.

In one aspect, the initial micronization is performed by mechanical grinding or shredding. In another aspect, micronization is performed by cryogenic grinding. In this aspect, the grinding jar containing the tissue is continually cooled with liquid nitrogen from the integrated cooling system before and during the grinding process. Thus, the sample is embrittled and volatile components are preserved. Moreover, the denaturing of proteins in the amnion, intermediate tissue layer, and/or chorion is minimized or prevented. In one aspect, the CryoMill manufactured by Retsch can be used in this aspect.

The selection of components used to make the molded, dehydrated amnion compositions described herein can vary depending upon the end-use of the amnion compositions. For example, placental tissue or individual components such as amnion, chorion, intermediate tissue layer, Wharton's jelly or any combination thereof can be admixed with one another and subsequently micronized. In another aspect, one or more tissue grafts composed of one or more placental tissue, amnion, chorion, intermediate tissue layers, or any combination thereof (i.e., laminates) can be micronized. In a further aspect, one or more tissue grafts composed of one or more amnion, chorion, intermediate tissue layers, or any combination can be admixed with amnion, chorion, intermediate tissue layer, or any combination thereof as individual components and subsequently micronized.

The amount of different components can vary depending upon the application of the molded, dehydrated placental composition. In one aspect, when the molded, dehydrated placental composition is composed of amnion (with or without the intermediate tissue layer) and intermediate tissue layer, the weight ratio of amnion to intermediate tissue layer is from about 10:1 to about 1:10, about 9:1 to about 1:1, about 8:1 to about 1:1, about 7:1 to about 1:1, about 6:1 to about 1:1, about 5:1 to about 1:1, about 4:1 to about 1:1, about 3:1 to about 1:1, about 2:1 to about 1:1, or about 1:1. In another aspect, when the molded, dehydrated placental tissue graft is composed of amnion (with or without the intermediate tissue layer) and chorion, the weight ratio of chorion to amnion is from about 10:1 to about 1:10, about 9:1 to about 1:1, about 8:1 to about 1:1, about 7:1 to about 1:1, about 6:1 to about 1:1, about 5:1 to about 1:1, about 4:1 to about 1:1, about 3:1 to about 1:1, about 2:1 to about 1:1, or about 1:1.

Separation of particle sizes can be achieved by fractionation of the micronized material in sterile water by forming a suspension of particles. The upper most portion of the suspension will contain predominantly the smallest particles and the lower most portion of the suspension will contain predominantly the heaviest particles. Fractionation leads to particle size separation and repeated fractionation will lead to separation of the micronized particles into varying sizes. The so separated particles can be recombined in the desired ratio of particle size as is most appropriate for making the molded placental tissue graft and the desired medical application. As one skilled in the art would appreciate, different sizes of the particles result in different density and cohesive mass of the molded placental composition, discussed below.

### Preparation of Molded Amnion Compositions and Compositions Thereof (Step 160)

This invention is based, in part, on the discovery that the disintegration rate in vivo of the molded compositions described herein can be preselected by accounting for the particle size of the dehydrated micronized particles. As shown in the examples below, altering particle size allows for predictable changes in the disintegration rate of the molded composition. Accordingly, this invention also provides for a method for producing a molded placental composition, including but not limited to a molded amnion composition, having a preselected disintegration rate in vivo, said method comprising molding an amnion composition under pressure wherein the particle size is adjusted prior to said molding so as to provide a molded amnion composition having a preselected disintegration rate. In one embodiment, the disintegration rate in vivo can be reduced (slowed) by decreasing the particle size of the micronized composition.

This invention is also based, in part, on the discovery that the strength and/or stiffness of the molded compositions described herein can be preselected by accounting for the particle size of the dehydrated micronized particles, the compression force used, and the rate at which the compression force is applied. As shown in the examples below, altering one or more of these parameters allows for predictable changes in the stiffness and/or strength of the molded composition. Accordingly, this invention also provides for a method for producing a molded placental composition having a preselected strength and/or stiffness, said method comprising molding an placental composition under pressure wherein one or more of the above parameters is adjusted prior to said molding so as to provide a molded placental composition having a preselected strength and/or stiffness. In one embodiment, the strength of the molded composition can be increased by decreasing the particle size of the micronized composition, while maintaining each of the other factors listed above. In one embodiment, the strength of the molded composition can be increased by increasing the compression force used. In one embodiment, the strength of the molded composition can be increased by decreasing the compression rate used.

The dehydrated micronized components, such as micronized amnion, and optionally chorion, intermediate tissue layer, and any combination thereof, when subjected to pressure preferably in a non-porous mold, form a desired shape and size defined by the mold. While a porous mold is less preferred, it is contemplated that such can be used in the methods of this invention if water or other solvents are allowed to escape during molding.

The compression force, compression rate, and number of compression cycles can vary during the formation of the molded, micronized placental composition. In one aspect, the compression force used to mold the micronized placenta is between 10 Newtons and 1000 Newtons. In a preferred embodiment, the compression force used to mold the micronized placenta is between 100 Newtons and 400 Newtons. The compression force can vary based on the intended use. For example, a use requiring greater strength and/or stiffness of the molded composition will require a greater force.

In one aspect, the compression rate used to mold the micronized placenta or placental tissue is between 0.001 mm/sec and 5 mm/sec. In a preferred embodiment, the compression rate used to mold the micronized placenta or placental tissue is between 0.008 mm/sec and 1.5 mm/sec. The compression rate can vary based on the intended use. For example, a use requiring greater strength and/or stiffness of the molded composition will require a slower rate.

The molded placental composition has a sufficient density and cohesive mass to maintain its size and shape at least until the molded placental composition is introduced to a subject. The cohesion of the molded placental composition is determined, in part, by the particle size of the micronized components. For example, dehydrated micronized components having larger particle size require higher compressive pressure and/or longer compression time to obtain a molded amnion composition having the same density as that of a molded placental composition composed of dehydrated micronized components having smaller particle size. In other words, for molded dehydrated amnion compositions obtained under the same compression condition, the compositions having larger particle size have less density and dissociate at a higher rate in comparison to the compositions having smaller particle size.

The particle size of the dehydrated micronized placental compositions, including but not limited to amnion compositions, also affects the release rate of the growth factors and other active molecules present in the composition. Without being bound by theory and with all other factors being equal, it is contemplated that smaller particle size creates a larger overall surface area of components within the composition. A larger surface area may result in an increased release of factors from the micronized placenta, such as amnion and/or other components, and/or a faster rate of release. Smaller particles are contemplated to allow for improved compressibility and increased strength. Molded, micronized placental compositions made with larger particles may disintegrate faster than those made with smaller particles. Therefore, the particle size of the micronized components can be optimized, thereby obtaining the molded placental composition having a desired cohesiveness, surface area, and desired end results when administered to a subject.

Optionally, one or more adhesives can be admixed with the micronized components prior to being introduced into the mold. Examples of such adhesives include, but are not limited to, fibrin sealants, cyanoacrylates, gelatin and thrombin products, polyethylene glycol polymer, albumin, and glutaraldehyde products. The adhesives used in the process should be dehydrated prior to being mixed with the micronized placental composition such that the mixture of adhesives and micronized placental composition has a sufficiently low water content to permit compression in a non-porous mold.

In addition to the placenta component, such as the amnion, the intermediate tissue layer, and/or the chorion, additional dehydrated components can be added to the composition prior to and/or after micronization. In one aspect, a dehydrated filler can be added. Examples of fillers include, but are not limited to, allograft pericardium, allograft acellular dermis, purified Type-1 collagen, human collagen, biocellulose polymers or copolymers, biocompatible synthetic polymer or copolymer films, purified small intestinal submucosa, bladder acellular matrix, cadaveric fascia, bone particles (including cancellous and cortical bone particles), or any combination thereof.

In another aspect, a dehydrated bioactive agent can be added to the composition prior to and/or after micronization. Examples of bioactive agents include, but are not limited to, naturally occurring growth factors sourced from platelet concentrates, either using autologous blood collection and separation products, or platelet concentrates sourced from expired banked blood; bone marrow aspirate; stem cells derived from concentrated human placental cord blood stem cells, concentrated amniotic fluid stem cells or stem cells grown in a bioreactor; or antibiotics. Upon application of the molded, dehydrated placental composition with bioactive agent to the region of interest, the bioactive agent is delivered to the region over time. Thus, the molded, dehydrated placental compositions described herein are useful as delivery devices of bioactive agents and other cosmetic agents when administered to a subject. Release profiles can be modified based on, among other things, the selection of the components used to make the molded placental composition as well as the size of the particles contained in the placental composition.

### Plasticizers

In yet another aspect, the micronized placental composition components are admixed with at least one plasticizer. One skilled in the art would select a suitable plasticizer based on the biocompatibility of the plasticizer, effect of plasticizer on the degradation or erosion rate of the amnion composition in vivo, effect of the plasticizer on the properties of the mixture to facilitate the molding/compression process, and/or effect of the plasticizer on the strength, flexibility, consistency, hydrophobicity and/or hydrophilicity of the composition. In some aspects, the plasticizer is dehydrated and/or micronized prior to being mixed with the micronized placental composition components such that the mixture of plasticizer and micronized placental composition components has a sufficiently low water content to permit compression in a non-porous mold.

The terms “plasticizer” and “plasticizing agent” can be used interchangeably in the present invention. A plasticizing agent can include any agent or combination of agents that can be added to modify the mechanical properties of the composition or a product formed from the composition.

Without intending to be bound by any theory or mechanism of action, plasticizers can be added, for example, to reduce crystallinity, lower the glass-transition temperature (Tg), or reduce the intermolecular forces between components within the composition, with a design goal that may include creating or enhancing a flow between components in the composition. The mechanical properties that are modified include, but are not limited to, Young's modulus, tensile strength, impact strength, tear strength, and strain-to-failure. A plasticizer can be monomeric, polymeric, co-polymeric, or a combination thereof, and can be added to a composition with or without covalent bonding. Plasticization and solubility are analogous to the extent that selecting a plasticizer involves considerations similar to the considerations in selecting a solvent such as, for example, polarity. Furthermore, plasticizers can also be added to a composition through covalent bonding that changes the molecular structure of the composition through copolymerization.

Examples of plasticizing agents include, but are not limited to, low molecular weight polymers such as, for example, single-block polymers, multi-block polymers, and copolymers; oligomers such as, for example, lactic acid oligomers including, but not limited to, ethyl-terminated oligomers of lactic acid; dimers of cyclic lactic acid and glycolic acid; small organic molecules; hydrogen bond forming organic compounds with and without hydroxyl groups; polyols such as low molecular weight polyols having aliphatic hydroxyls; alkanols such as butanols, pentanols and hexanols; sugar alcohols and anhydrides of sugar alcohols; polyethers such as poly(alkylene glycols); esters such as citrates, phthalates, sebacates and adipates; polyesters; aliphatic acids; saturated and unsaturated fatty acids; fatty alcohols; cholesterol; steroids; phospholipids such as, for example, lecithin; proteins such as animal proteins and vegetable proteins; oils such as, for example, the vegetable oils and animal oils; silicones; acetylated monoglycerides; diglycerides; triglycerides; amides; acetamides; sulfoxides; sulfones; pyrrolidones; oxa acids; diglycolic acids; and any analogs, derivatives, copolymers and combinations thereof.

In some embodiments, the plasticizers include, but are not limited to other polyols such as, for example, caprolactone diol, caprolactone triol, sorbitol, erythritol, glucidol, mannitol, sorbitol, sucrose, and trimethylol propane. In other embodiments, the plasticizers include, but are not limited to, glycols such as, for example, ethylene glycol, diethylene glycol, Methylene glycol, tetraethylene glycol, propylene glycol, butylene glycol, 1,2-butylene glycol, 2,3-butylene glycol, styrene glycol, pentamethylene glycol, hexamethylene glycol; glycol-ethers such as, for example, monopropylene glycol monoisopropyl ether, propylene glycol monoethyl ether, ethylene glycol monoethyl ether, and diethylene glycol monoethyl ether; and any analogs, derivatives, copolymers and combinations thereof.

In other embodiments, the plasticizers include, but are not limited to esters such as glycol esters such as, for example, diethylene glycol dibenzoate, dipropylene glycol dibenzoate, methylene glycol caprate-caprylate; monostearates such as, for example, glycerol monostearate; citrate esters; organic acid esters; aromatic carboxylic esters; aliphatic dicarboxylic esters; fatty acid esters such as, for example, stearic, oleic, myristic, palmitic, and sebacic acid esters; triacetin; poly(esters) such as, for example, phthalate polyesters, adipate polyesters, glutate polyesters, phthalates such as, for example, dialkyl phthalates, dimethyl phthalate, diethyl phthalate, isopropyl phthalate, dibutyl phthalate, dihexyl phthalate, dioctyl phthalate, diisononyl phthalate, and diisodecyl phthalate; sebacates such as, for example, alkyl sebacates, dimethyl sebacate, dibutyl sebacate; hydroxyl-esters such as, for example, lactate, alkyl lactates, ethyl lactate, butyl lactate, allyl glycolate, ethyl glycolate, and glycerol monostearate; citrates such as, for example, alkyl acetyl citrates, triethyl acetyl citrate, tributyl acetyl citrate, trihexyl acetyl citrate, alkyl citrates, triethyl citrate, and tributyl citrate; esters of castor oil such as, for example, methyl ricinolate; aromatic carboxylic esters such as, for example, trimellitic esters, benzoic esters, and terephthalic esters; aliphatic dicarboxylic esters such as, for example, dialkyl adipates, alkyl allylether diester adipates, dibutoxyethoxyethyl adipate, diisobutyl adipate, sebacic esters, azelaic esters, citric esters, and tartaric esters; and fatty acid esters such as, for example, glycerol, mono- di- or triacetate, and sodium diethyl sulfosuccinate; and any analogs, derivatives, copolymers and combinations thereof.

In other embodiments, the plasticizers include, but are not limited to ethers and polyethers such as, for example, poly(alkylene glycols) such as poly(ethylene glycols) (PEG), polypropylene glycols), and poly(ethylene/propylene glycols); PEG derivatives such as, for example, methoxy poly(ethylene glycol) (mPEG); and ester-ethers such as, for example, diethylene glycol dibenzoate, dipropylene glycol dibenzoate, and triethylene glycol caprate-caprylate; and any analogs, derivatives, copolymers and combinations thereof.

In other embodiments, the plasticizers include, but are not limited to, amides such as, for example, oleic amide, erucic amide, and palmitic amide; alkyl acetamides such as, for example, dimethyl acetamide; sulfoxides such as for example, dimethyl sulfoxide (DMSO); pyrrolidones such as, for example, n-methyl pyrrolidone; sulfones such as, for example, tetramethylene sulfone; acids such as, for example, oxa monoacids, oxa diacids such as 3,6,9-trioxaundecanedioic acid, polyoxa diacids, ethyl ester of acetylated citric acid, butyl ester of acetylated citric acid, capryl ester of acetylated citric acid, and diglycolic acids such as dimethylol propionic acid; and any analogs, derivatives, copolymers and combinations thereof.

In other embodiments, the plasticizers include, but are not limited to vegetable oils including, but not limited to, epoxidized soybean oil; linseed oil; castor oil; coconut oil; fractionated coconut oil; epoxidized tallates; and esters of fatty acids such as stearic, oleic, myristic, palmitic, and sebacic acid; essential oils including, but not limited to, angelica oil, anise oil, arnica oil, aurantii aetheroleum, valerian oil, basilici aetheroleum, bergamot oil, savory oil, bucco aetheroleum, camphor, cardamomi aetheroleum, cassia oil, chenopodium oil, chrysanthemum oil, cinae aetheroleum, citronella oil, lemon oil, citrus oil, costus oil, curcuma oil, carlina oil, elemi oil, tarragon oil, eucalyptus oil, fennel oil, pine needle oil, pine oil, filicis, aetheroleum, galbanum oil, gaultheriae aetheroleum, geranium oil, guaiac wood oil, hazelwort oil, iris oil, hypericum oil, calamus oil, chamomile oil, fir needle oil, garlic oil, coriander oil, carraway oil, lauri aetheroleum, lavender oil, lemon grass oil, lovage oil, bay oil, lupuli strobuli aetheroleum, mace oil, marjoram oil, mandarine oil, melissa oil, menthol, millefolii aetheroleum, mint oil, clary oil, nutmeg oil, spikenard oil, clove oil, neroli oil, niaouli, olibanum oil, ononidis aetheroleum, opopranax oil, orange oil, oregano oil, orthosiphon oil, patchouli oil, parsley oil, petit-grain oil, peppermint oil, tansy oil, rosewood oil, rose oil, rosemary oil, rue oil, sabinae aetheroleum, saffron oil, sage oil, sandalwood oil, sassafras oil, celery oil, mustard oil, serphylli aetheroleum, immortelle oil, fir oil, teatree oil, terpentine oil, thyme oil, juniper oil, frankincense oil, hyssop oil, cedar wood oil, cinnamon oil, and cypress oil; and other oils such as, for example, fish oil; and any analogs, derivatives, copolymers and combinations thereof.

It should be appreciated that, in some embodiments, one of skill in the art may select one or more particular plasticizing agents in order to exclude any one or any combination of the above-described plasticizing agents.

In some embodiments, the plasticizing agent can include a component that is water-soluble. In other embodiments, the plasticizing agent can be modified to be water-soluble. In some embodiments, the plasticizing agent can include a component that is lipid-soluble. In other embodiments, the plasticizing agent can be modified to be lipid-soluble. Any functional group can be added to modify the plasticizer's behavior in a solvent such as, for example, body fluids that are present in vivo.

### Cross-Linking

In a further aspect, the in vivo degradation or erosion rate of the molded placental composition, as well as the density and cohesiveness of the dehydrated micronized components, can be modified, for example, by cross-linking. For example, the amnion can be cross-linked with other components, such as the intermediate tissue layer, the chorion, or a second amnion tissue. For example, a cross-linking agent can be added to the composition (e.g., placenta, amnion, chorion, intermediate tissue layer, other component, or any combination thereof as individual components and/or as tissue grafts) prior to and/or after micronization. In general, the cross-linking agent is nontoxic and non-immunogenic. When the components are treated with the cross-linking agent, the cross-linking agent can be the same or different. In one aspect, the placenta, the amnion, the intermediate tissue layer, the chorion, and/or other component can be treated separately with a cross-linking agent or, in the alternative, the placenta, the amnion, the intermediate tissue layer, the chorion, and/or other component can be treated together with the same cross-linking agent. In certain aspects, the placenta, the amnion, the intermediate tissue layer, the chorion, and/or other component can be treated with two or more different cross-linking agents. The conditions for treating the amnion and/or other components can vary. In other aspects, the placenta, the amnion, the intermediate tissue layer, the chorion, and/or other components can be micronized, and the micronized composition can subsequently be treated with a cross-linking agent. In one aspect, the concentration of the cross-linking agent is from about 0.1 M to about 5 M, about 0.1 M to about 4 M, about 0.1 M to about 3 M, about 0.1 M to about 2 M, or about 0.1 M to about 1 M. Preferably, the components are cross-linked prior to dehydration such that the cross-linked components have a sufficiently low water content to permit compression or molding in a non-porous mold.

In certain aspects, the molded, dehydrated placental composition can be treated with the cross-linking agent. Preferably, the placental composition is subjected to gas/fume cross-linking prior to compression and before or after micronization such that the water content of the placental composition is maintained at a low level, e.g., less than about 20%, less than about 15%, less than about 10%, or less than about 5%. The cross-linking agent generally possesses two or more functional groups capable of reacting with proteins to produce covalent bonds. In one aspect, the cross-linking agent possesses groups that can react with amino groups present on the protein. Examples of such functional groups include, but are not limited to, hydroxyl groups, substituted or unsubstituted amino groups, carboxyl groups, and aldehyde groups. In one aspect, the cross-linker can be a dialdehyde such as, for example, glutaraldehyde. In another aspect, the cross-linker can be a carbodiimide such as, for example, (N-(3-dimethylaminopropyl)-N′-ethyl-carbodiimide (EDC). In other aspects, the cross-linker can be an oxidized dextran, p-azidobenzoyl hydrazide, N-[alpha-maleimidoacetoxy]succinimide ester, p-azidophenyl glyoxal monohydrate, bis-[beta-(4-azidosalicylamido)ethyl]disulfide, bis-[sulfosuccinimidyl]suberate, dithiobis[succinimidyl]propionate, disuccinimidyl suberate, and 1-ethyl-3-[3-dimethylaminopropyl]carbodiimide hydrochloride, a bifunctional oxirane (OXR), ethylene glycol diglycidyl ether (EGDE), nordihydroguaiaretic acid (NDGA).

In one aspect, sugar is the cross-linking agent, where the sugar can react with proteins present in the placental component, such as amnion and/or other components to form a covalent bond. For example, the sugar can react with proteins by the Maillard reaction, which is initiated by the nonenzymatic glycosylation of amino groups on proteins by reducing sugars and leads to the subsequent formation of covalent bonds. Examples of sugars useful as cross-linking agents include, but are not limited to, D-ribose, glycerose, altrose, talose, ertheose, glucose, lyxose, mannose, xylose, gulose, arabinose, idose, allose, galactose, maltose, lactose, sucrose, cellibiose, gentibiose, melibiose, turanose, trehalose, isomaltose, or any combination thereof.

The cosmetic compositions described herein can be administered in a number of ways depending on whether local or systemic treatment is desired, and on the area to be treated. In one aspect, administration can be by injection. In other aspects, the molded dehydrated placental composition can be formulated to be applied internally to a subject. In other aspects, the molded dehydrated placental composition can be applied topically, subdermally or subcutaneously.

### III. Applications of Compositions Comprising Molded Micronized Amnion

The compositions comprising molded dehydrated amnion described herein have numerous cosmetic applications. The particle size, density, strength, disintegration rate, and other properties of the molded, micronized placental compositions described herein can be modified based on the intended use. For example, a molded, micronized placental composition with smaller particle size and/or higher density can be used for an application requiring a longer disintegration time and/or prolonged release of factors. Conversely, a molded, micronized placental composition with larger particle size and/or lower density can be used for an application requiring a shorter disintegration time and/or more rapid release of factors.

The molded placental compositions described herein are useful in a variety of cosmetic treatments including, but not limited to, remodeling, filling or reconstruction of soft tissues, the treatment of wrinkles, creases and scars, burns, ulcers, soft tissue augmentation, facial lipoatrophy, as an analgesic and anti-inflammatory. This discussion of possible uses is not intended to be exhaustive and many other embodiments exist.

In some embodiments, the injectable nature of the compositions enables non-invasive, percutaneous delivery through a needle, syringe, injection syringe, cannula, trocar, or any other suitable applicator. Small diameter needles (e.g., 27 G) can be used, which is a valuable attribute when treating facial wrinkles and folds, sleep apnea, nasolabial folds, facial fat loss (lipoatrophy), vocal cord insufficiency, craniofacial augmentation and radiographic tissue marking.

### Cosmetic Applications

Human skin is a composite material of the epidermis and the dermis. The outermost layer of the epidermal layer of the skin is the stratum corneum. Beneath the stratum corneum layer is the epidermis. Below the epidermis is the outermost layer of the dermis called the papillary dermis, followed by the reticular dermis and the subcutaneous layer.

The skin serves many functions including protection, absorption, pigmentogenesis, sensory perception, secretion, excretion, thermoregulation, and regulation of immunological processes. These skin functions are negatively affected, for example, by aging, excessive sun exposure, smoking, trauma, and/or environmental factors, which cause structural changes in the skin and can result in impairment of the barrier function of the skin and a decreased turnover of epidermal cells. Damaged collagen and elastin lose the ability to contract properly, which results in skin wrinkling and surface roughness. Wrinkles are modifications of the skin that are typically associated with cutaneous aging and develop preferentially on sun-exposed skin. As aging progresses, the face, as well as other areas of the body, begin to show the effects of gravity, sun exposure and years of, e.g., facial muscle movement, such as smiling, chewing and squinting. As the skin ages or becomes unhealthy, it acquires wrinkles, sags, and stretch marks, it roughens, and it has a decreased ability to synthesize Vitamin D. Aged skin also becomes thinner and has a flattened dermoepidermal interface because of the alterations in collagen, elastin, and glycosaminoglycans. Typically, aging skin can be characterized by decreased thickness, elasticity, and adherence to underlying tissue.

Damage to the skin due to aging, environmental factors, exposure to the sun and other elements, such as weight loss, child bearing, disease (e.g., acne and cancer) and surgery often results in skin contour deficiencies and other skin anomalies. In order to correct perceived contour deficiencies and other perceived anomalies of the skin, people often resort to cosmetic surgery, such as face lifts and skin tucks. Cosmetic surgery, however, is generally expensive, invasive, has the potential of leaving scars in the areas of operation, and may affect normal biological and physiological functions. Thus, there remains a need for alternative therapies.

The invention provides methods for skin augmentation in a patient. In one embodiment, a method for skin augmentation in a patient comprises injecting or otherwise administering a molded placental composition of the invention to an area of the face or body of a patient perceived to be in need of augmenting, wherein the area of the face or body of the patient is augmented as compared to the area prior to administration of the molded placental composition. “Skin augmentation” in the context of the present invention refers to any change of the natural state of a patient's (e.g., a human's) skin and related areas due to external acts or effects. Non-limiting areas of the skin that may be changed by skin augmentation include the epidermis, dermis, subcutaneous layer, fat, arrector pili muscle, hair shaft, sweat pore, sebaceous gland, or a combination thereof.

Without being bound by theory, it is contemplated that the molded, dehydrated placental composition of the current invention will contribute to augmentation in both the short- and long-term. That is to say, administration of the composition to an area to be augmented may result in short-term augmentation by the presence of the molded composition. Further, the placental component can recruit stem cells to populate the area and lead to long-term augmentation.

In some embodiments, methods of the invention comprise injecting or otherwise administering a molded placental composition of the invention to a patient for the treatment of crow's feet, nasolabial folds (“smile lines”), marionette lines, glabellar folds (“frown lines”), or a combination thereof. A molded placental composition of the invention can help fill in lines, creases, and other wrinkles and restore a smoother, more youthful-looking appearance. A molded placental composition of the invention can be used alone or in conjunction with one or more additional injectable compositions, a resurfacing procedure, such as a laser treatment, or a recontouring procedure, such as a facelift.

In one embodiment, a molded placental composition of the invention may also be used to augment creased or sunken areas of the face and/or to add or increase the fullness to areas of the face and body of a patient. The areas of the face and/or body requiring augmentation may be the result of, e.g., aging, trauma, disease, sickness, environmental factors, weight loss, child birth, or a combination thereof. Non-limiting examples of an area of the face or body of a patient where a molded placental composition of the invention may be injected or otherwise administered include the undereye, temple, upper malar, sub malar, chin, lip, jawline, forehead, glabella, outer brow, cheek, area between upper lip and nose, nose (such as the bridge of the nose), neck, buttocks, hips, sternum, or any other part of the face or body, or a combination thereof.

In further aspects, a molded placental composition of the invention may be used to treat skin deficiencies including, but not limited to, wrinkles, depressions or other creases (e.g., frown lines, worry lines, crow's feet, marionette lines), stretch marks, internal and external scars (such as scars resulting from injury, wounds, accidents, bites, or surgery), or combinations thereof. In some embodiments, a molded placental composition of the invention may be used for the correction of, for example, “hollow” eyes, visible vessels resulting in dark circles, as well as visible tear troughs. A molded placental composition of the invention may also be used, for example, for correction of the undereye after aggressive removal of undereye fat pads from lower blepharoplasty or correction of the lower cheek after aggressive buccal fat extraction or natural loss. In one embodiment, a molded placental composition of the invention may be used to correct the results of rhinoplasty, skin graft or other surgically-induced irregularities, such as indentations resulting from liposuction. In other embodiments, a molded placental composition of the invention may be used for the correction of facial or body scars (e.g., wound, chicken pox, or acne scars). In some embodiments, a molded placental composition of the invention is injected or otherwise administered into a patient for facial reshaping. Facial reshaping using the methods of the invention may be completed in a patient with neck laxity, or having a gaunt face, long face, bottom-heavy face, asymmetrical face, a chubby face, or having a face with localized fat atrophy, a midface retrusion, sunken eyes, and/or any combinations thereof.

In one embodiment, the methods of the invention comprise injecting or otherwise administering a molded placental composition of the invention to a patient for the treatment of a perceived skin deficiency, such as skin deficiency caused by a disease or illness, such as cancer or acne. The deficiency can be the direct or indirect result of the disease or illness. For example, a skin deficiency can be caused by a disease or illness or can be caused by a treatment of a disease or illness.

The following abbreviations are used in this application:

| Abbreviation | Definition |
|---|---|
| cm | centimeters |
| ° C. | degrees Celsius |
| g/cm³ | grams per cubic centimeter |
| MPa | megapascal |
| μm | micrometer |
| mg | milligram |
| ml | milliliter |
| mm | millimeter |
| mm/min | millimeter per minute |
| mm/sec | millimeter per second |
| M | mole |
| N | Newton |
| rpm | rotation per minute |
| NaCl | sodium chloride |

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### Preparation of Micronized Composition

Amnion/chorion tissue grafts used here to produce the micronized particles were produced by the process described in US 2008/0046095, which is incorporated by reference in its entirety. Tissue grafts (4 cm×3 cm) and two 9.5 mm steel grinding balls were placed in 50 mL vials and the vials subsequently sealed. The vials were placed in the Cryo-block, and the Cryo-block was placed in a Cryo-rack. The Cryo-rack was placed into a liquid nitrogen holding Dewar. Tissue samples were subjected to vapor phase cooling for no more than 30-60 minutes. The Cryo-rack was removed from the Dewar, and the Cryo-block was removed from the Cryo-rack. The Cryo-block was placed into the Grinder (SPEX Sample Prep GenoGrinder 2010) and set at 1,500 rpm for 20 minutes. After 20 minutes has elapsed, the tissue is inspected to ensure micronization. If necessary, the tissue can be placed back into the Dewar for an additional 30-60 minutes, and moved to the grinder for an additional 20 minutes to ensure sufficient micronization. Once the tissue is sufficiently micronized it is sorted using a series of American Standard ASTM sieves. The sieves were placed in the following order: 355 μm, 300 μm, 250 μm, 150 μm, and 125 μm. The micronized material was transferred from the 50 mL vials to the 355 μm sieve. Each sieve was agitated individually in order to thoroughly separate the micronized particles. Once the micronized particles have been effectively separated using the sieves, the micronized particles having particle sizes of 355 μm, 300 μm, 250 μm, 150 μm, and 125 μm were collected in separate vials.

### Example 2

### Preparation of Molded Placental Tissue Composition Comprising Micronized Particles

A mold having a female half and a male half in a crescent moon shape is used in preparing molded dehydrated placental tissue composition. The female half is loaded with 10 mg micronized amnion/chorion particles prepared in Example 1. Once the male half is in place to contain the microparticles, the mold is subjected to a suitable pressure for a suitable period of time to obtain the molded placental tissue composition having the size and shape of the mold.

In one embodiment, the mold is a non-porous mold and the water content of the dehydrated placental tissue is less than about 25% weight/weight. In another embodiment, the water content is greater 25% weight/weight and a porous mold is used to allow excess water to be removed. Alternatively, a small volume (e.g. 10 weight percent or less) of a biocompatible organic solvent can be added to the dehydrated placental tissue so that during compression, the solvent vaporization will lead to small voids within the composition.

### Example 3

### Effect of Particle Size on Molded Placental Tissue Composition Properties

Micronized placental tissue was inserted into a mold having a diameter of 1 mm and compressed with a force of 400 N at a rate of 1 mm/sec. Three sizes of micronized particles were tested: greater than about 250 μm, between about 75 μm and about 150 μm, and less than about 75 μm approximate diameter. The stress at failure and Young's Modulus were measured. Compression tests were performed at a rate of 0.5 mm/min.

The molded, micronized compositions (“plugs”) were further tested for the rate of disintegration and degradation. Molded, micronized amnion plugs were incubated in saline at 37° C. for increasing lengths of time.

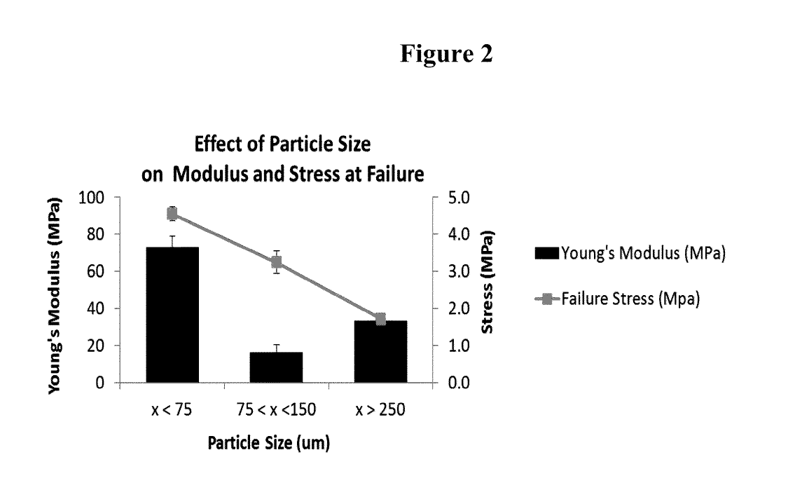

Results: The effect of particle size on modulus and stress at failure is represented in FIG. 2. Molded compositions comprising a particle size of less than 75 μm consistently withstood the highest compressive load with minimal strain, resulting in the highest Young's Modulus. Plugs made with larger particle sizes disintegrated faster in saline than those made with smaller particle sizes. Plugs maintained shape and form for at least 6 weeks in saline at 37° C. Incubation in saline resulted in swelling of plugs. An increase in plug length by 240% was observed after 1 hour in saline.

### Example 4

### Effect of Compression Force, Rate and Number of Cycles on Molded Placental Tissue Composition Properties

Micronized placental tissue (particle size less than about 75 μm) was inserted into a mold having a diameter of 1 mm and compressed. The effect of compression force during formation of the molded amnion composition was tested at a compression rate of 1 mm/sec. The effect of the rate of compression during formation of the molded amnion composition was tested at a compression force of 400 N. The effect of increasing cycles of compression during formation of the molded amnion composition was tested at a compression force of 400 N and compression rate of 1 mm/sec. The stress at failure and Young's Modulus were measured. Compression tests were performed at a rate of 0.5 mm/min.

The molded, micronized compositions were further tested for the rate of disintegration and degradation. Molded, micronized tissue plugs were incubated in saline at 37° C. for increasing lengths of time.

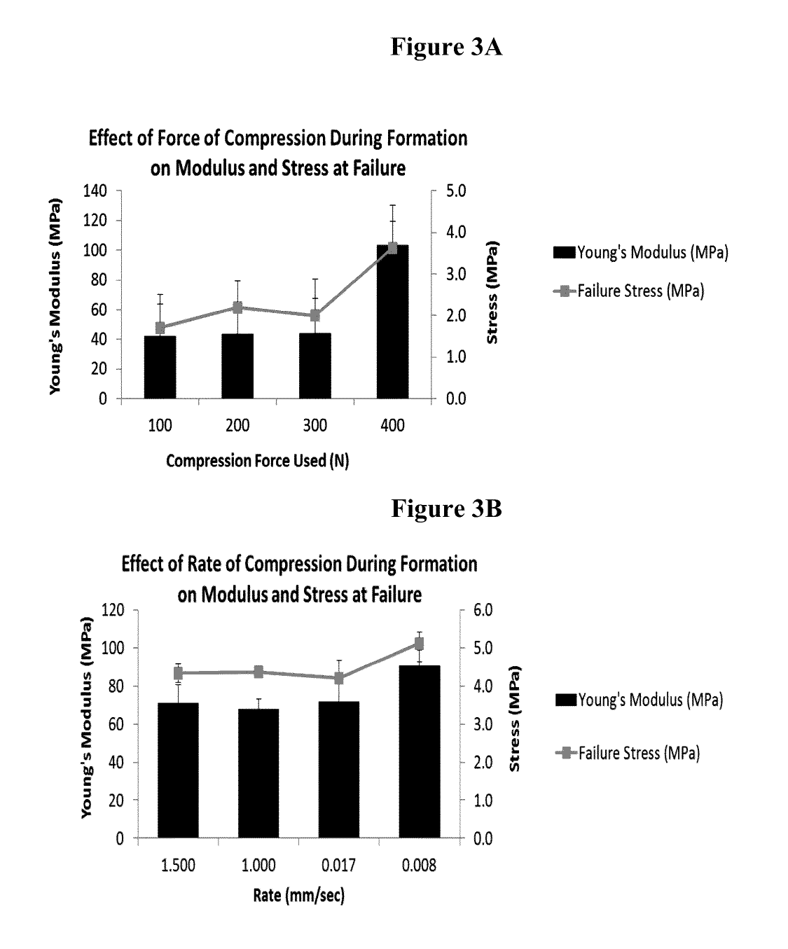

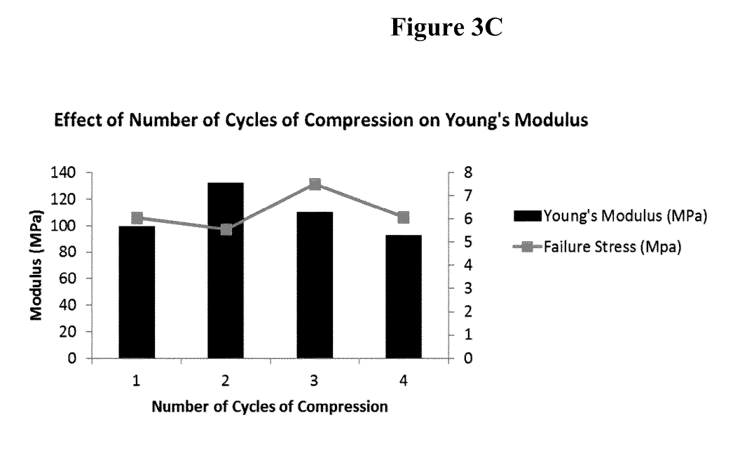

Results: The effect of compression force on modulus and stress at failure is represented in FIG. 3A. Plugs compressed with 400 N withstood the highest compressive load with minimal strain, resulting in the highest Young's Modulus. Plugs made with different compression forces did not display significantly different disintegration profiles. However, plugs compressed via machine maintained shape longer than those compressed manually.

The effect of compression rate on modulus and stress at failure is represented in FIG. 3B. Plugs compressed at 0.008 mm/sec (0.5 mm/min) withstood the highest compressive load with minimal strain, resulting in the highest Young's Modulus. Plugs made with different compression rates did not display significantly different disintegration profiles in saline.

The effect of the number of cycles of compression on modulus and stress at failure is represented in FIG. 3C. Plugs subjected to two cycles of compression of 400 N each resulted in the stiffest plug (highest Young's Modulus). Plugs subjected to three cycles of compression of 400 N each resulted in the strongest plug (highest stress at failure).

### Example 5

### In Situ Testing of Molded Placental Tissue Composition

Molded, micronized amnion plugs were inserted into a syringe or trocar and placed into porcine shoulder muscle tissue. The tissue was incubated in saline and cut open and examined at 30 minutes, 3 hours, or 3 days.

Results: The molded, micronized amnion plugs maintained shape at each time point. Plug length expanded up to 60% within the tissue after 3 days.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

A detailed description of suitable cross-linking agents and procedures is provided in U.S. patent application Ser. No. 13/815,747 filed on Mar. 15, 2013, which application is incorporated herein by reference in its entirety.

A detailed description of micronized placental tissue is provided in U.S. patent application Ser. No. 13/815,784 filed Mar. 15, 2013, and Ser. No. 13/963,984, filed on Aug. 9, 2013, which applications are incorporated herein by reference in their entireties.

What is claimed:

1. A cosmetic method for altering the appearance of a patient which method comprises identifying a perceived defect in the patient and altering said defect by placing a placental composition in or adjacent said defect in a manner to alter the appearance of the defect, wherein the placental composition comprises molded, micronized placental tissue.

2. The cosmetic method according to claim 1, wherein said cosmetic method is selected from the group consisting of a wrinkle reduction, lip augmentation, collagen replacement treatment, and facial augmentation.

3. The cosmetic method of claim 1 wherein said placental composition comprises molded micronized amnion and optionally micronized biocompatible polymer.

4. The cosmetic method of claim 3, wherein said micronized biocompatible polymer is a plasticizing polymer.

5. A molded placental composition comprising micronized placental tissue which further comprises a biocompatible plasticizing polymer.

6. The molded placental composition of claim 5, wherein the micronized placental tissue is cross-linked with a biocompatible cross-linking agent.

7. The molded placental composition of claim 6, wherein the biocompatible plasticizing polymer is cross-linked with a biocompatible cross-linking agent.

8. A cosmetic method for altering the appearance of a patient which method comprises administering a molded placental composition to a subject, wherein said molded placental composition has a defined size and shape comprising micronized placental tissue, and wherein said composition has a sufficient density and cohesiveness to maintain its size and shape for a defined period of time ex vivo and in vivo.

9. A method for producing a molded placental composition having a preselected disintegration rate and/or strength, said method comprising adjusting the compression force, compression rate, density and/or particle size such that the resulting molded placental composition has the preselected disintegration rate and/or strength.

10. The method of claim 9, wherein the particle size is greater than about 250 μm.

11. The method of claim 9, wherein the particle size is between about 75 μm and about 150 μm.

12. The method of claim 9, wherein the particle size is less than about 75 μm.

13. A method for producing a molded placental composition having a preselected strength and/or stiffness, said method comprising adjusting the compression force, compression rate, density and/or particle size such that the resulting molded placental composition has the preselected strength and/or stiffness.
